(19) United States
(12) Patent Application Publication
(10) Pub. No.: US 2019/0209609 A1
(43) Pub. Date: Jul. 11, 2019

(54) METHODS AND COMPOUNDS FOR PH CONTROLLED ANTICOAGULANT USING PHOSPHATES

(71) Applicant: FORMTECH SOLUTIONS INC., College Station, TX (US)

(72) Inventors: Peter Oberacker, Schenevus, NY (US); Ron Wheeler, College Station, TX (US)

(21) Appl. No.: 15/756,360

(22) PCT Filed: Sep. 29, 2017
(86) PCT No.: PCT/US17/54223

§ 371 Date: Feb. 28, 2018

Related U.S. Application Data
(60) Provisional application No. 62/401,553, filed on Sep. 29, 2016.

(51) Int. Cl. A61K 33/42; A61K 47/02

(52) U.S. Cl. CPC A61K 33/42; A22B 5/04; A61K 47/02

(57) ABSTRACT

This disclosure provides methods and compounds for pH controlled anticoagulant materials using phosphates. At least one phosphate and at least one alkalizing processing aid are combined in an anticoagulant compound that may be applied in an aqueous solution to acidifying animal blood to substantially neutralize the animal blood for further processing, such as fractionization.

### BACKGROUND

The disclosure relates generally to anticoagulant compounds for use in blood processing and, more specifically, anticoagulant compounds for use in slaughterhouses.

Anticoagulants are used in slaughterhouse operations to enable recovering of blood from the work area. Blood is collected from the area where animals are slaughtered and bled. The goal is to avoid the accumulation of coagulated blood, which is more difficult to clean and may create safety hazards, and to recover blood for other uses.

Blood from slaughtered animals may be collected and used for additional products or other purposes. While whole blood may be used for low-value purposes like fertilizer or feed supplements, there is a growing market for processed animal blood constituents, such as plasma and red blood cells. For example, dried animal plasma proteins may be processed to make protein additives for use in a variety of products, including food products for human consumption, and dried animal red blood cells are used as an ingredient in dark breads and other food stuffs in some countries. Processed animal blood components may also be used in a variety of animal feed products, such as calf milk replacer products, and have other applications in medical, veterinary, health & beauty, and other products.

Unless prevented, blood from slaughtered animals quickly coagulates after being exposed to air. Water alone can be used to wash blood collection areas. However, the volume of water dilutes the blood products of interest, increases processing time and cost, and may lead to lower yields and/or lower quality blood products.

Animal blood from slaughterhouses may be collected, stored, and then processed into desired fractions or products. Some slaughterhouses include mechanical features for capturing and/or moving animal blood during the slaughter process. For example, basins, troughs, or other catch features may be positioned to receive blood, and then channels, drains, or conduits may lead to blood collection tanks or other apparatus. In some slaughterhouses, an anticoagulant compound is added to the blood as soon as possible after it has left the animal. For example, an anticoagulant may be sprayed continuously on the surface of the catch feature to mix with the blood as it drains from the animal. The anticoagulant keeps the blood in liquid form until the desired processing can be done, such as separation, purification, concentration, drying, and/or other processing steps. Treated blood in liquid form can then flow to suitable collection or storage vessels, or may be pumped or otherwise moved to different locations for processing. Treated blood may be processed into products according to a number of processing methods. Common processing techniques may include filtration, centrifuge, spray drying, freeze drying, concentration, and purification.

While the use of anticoagulants may increase the yield, uses, and value of animal blood and its constituents, the cost of the anticoagulant and the impact it has on the desired constituents is highly relevant. The purchase and handling costs of the anticoagulant cannot outweigh the value of the resulting products and reducing purchase and handling costs may directly improve the business case and profits from blood processing. In some facilities, 4,000 gallons or more of anticoagulant may be used every week. Storing, shipping, handling, mixing, pumping, and administering anticoagulants can represent significant costs. Effective volumes and yields based on anticoagulant properties are also highly relevant to cost. In particular, coagulated blood in the treated blood may result in added difficulties and costs in downstream processing from clogged filters, increased adhesion to surfaces, and accumulation. In addition, cell fragmentation and other destruction of blood constituents can negatively impact the quantity and quality of product yields.

A condition of slaughter (rigor) is a change from an aerobic condition to an anaerobic one. This results in the accumulation of lactic acid and a lowering of pH, generally from a relatively neutral state (˜7.0) to an acidic state (5.5 or lower). The lower pH causes the denaturing of the cell wall, which contributes to rupturing of the cell and creates lower yield opportunity and may impact other quality measures of resulting blood-based products. Pre-rigor pH is generally slightly basic, generally around 7.35-7.45.

Some anticoagulant compounds include Heparin, Alsever's solution (dextrose, sodium citrate, citric acid, sodium chloride), EDTA (ethylenediaminetetraactic acid), sodium citrate, and additives and combinations thereof.

### SUMMARY

A first aspect of this disclosure provides at least one phosphate in a percent dry mix of forty percent or less. At least one alkalizing processing aid is mixed with the phosphate to make up the balance of the anticoagulant compound in proportion to the phosphate. Where animal blood to be processed may have begun acidification, the concentration of anticoagulant compound may be calculated to return post-rigor animal blood to a substantially neutral pH.

A second aspect of this disclosure provides a method for a pH controlled anticoagulant compound using phosphates. At least one phosphate is selected. At least one alkalizing processing aid is selected. The selected phosphate and alkalizing processing aid are mixed to form an anticoagulant compound. The phosphate is forty percent or less of the anticoagulant compound and the alkalizing processing aid makes up the balance of the anticoagulant compound. A concentration of the anticoagulant compound calculated to return post-rigor animal blood that has begun acidification to a substantially neutral pH is selected.

A third aspect of this disclosure provides a method of producing fractionated animal blood in a meat processing facility. Acidifying animal blood is collected from an animal carcass. An anticoagulant compound is applied to the animal blood. The anticoagulant compound comprises an alkalizing processing aid and a phosphate in an aqueous solution with water. The acidic pH of the animal blood prior to applying the anticoagulant compound is substantially neutralized by the anticoagulant compound. The animal blood is fractionated into a cellular fraction and a liquid fraction. The liquid fraction is at least 53% of the fractionated animal blood.

The illustrative aspects of the present disclosure are arranged to solve the problems herein described and/or other problems not discussed.

### BRIEF DESCRIPTION OF THE DRAWINGS

These and other features of this disclosure will be more readily understood from the following detailed description of the various aspects of the disclosure taken in conjunction with the accompanying drawings that depict various embodiments of the disclosure, in which:

FIG. 1 shows an example method of making an anticoagulant compound according to embodiments of the disclosure.

FIG. 2 shows an example method of using an anticoagulant compound according to embodiments of the disclosure.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

In some embodiments, a stabilization additive, such as an anticoagulant compound, may be used prevent blood from coagulating and/or degrading from the time it is collected from the carcass of an animal in a slaughterhouse or other meat processing facility, until it can be further processed into one or more blood products, at the same facility or elsewhere. Stabilization additives may be delivered as an aqueous solution, such as an aqueous solution of an anticoagulant compound, to improve the ease of applying and mixing the stabilization additive with collected animal blood. In some embodiments, the stabilization additive both introduces one or more factors to prevent coagulation while simultaneously adjusting the pH level of the treated animal blood from an acidic and/or acidifying state back to near pre-rigor levels. For example, while the blood of a live animal is typically near neutral or slightly basic with a pH of 7-7.5, acidifying blood from an animal carcass may quickly fall well below the neutral pH of 7 and may approach a pH of 5.5 or lower. The stabilization additive may arrest the acidification of the collected animal blood and substantially neutralize treated animal blood, returning it to substantially neutral with a pH in the range of 6.5 to 7.5 and, in some embodiments, preferably on the slightly basic side of neutral at greater than 7 and less than 7.5. The use of an anticoagulant that adjusts pH to substantially neutral may stabilize the blood, prevent degradation of cell walls and other proteins, and allow higher yielding of both red and white blood cells while improving the overall color of plasma in fractionated blood. In general, the improvement of color means producing plasma that is lighter in color and tending toward translucent white to clear.

In some embodiments, the anticoagulant compound is generally comprised of one or more selected processing aids along with one or more selected phosphates. These processing aids and phosphates may be acquired in powder, solution, or other form and combined mechanically and/or chemically into the anticoagulant compound. For example, a powdered processing aid and a powdered phosphate may be mixed in desired proportions through stirring, tumbling, shaking, sifting, or other powder mixing methods. In some embodiments, the anticoagulant compound may consist entirely of the processing aid(s) and the phosphate(s) with no other ingredients and commercially reasonable purity (lacking substantial contaminants). In some embodiments, the anticoagulant compound may substantially consist of only the processing aid(s) and the phosphate(s), with the only other ingredients being non-functional or entirely unrelated to the purpose of the anticoagulant compound, namely prevention of clotting, slowing degradation of cells and proteins, and regulating the pH of the treated animal blood.

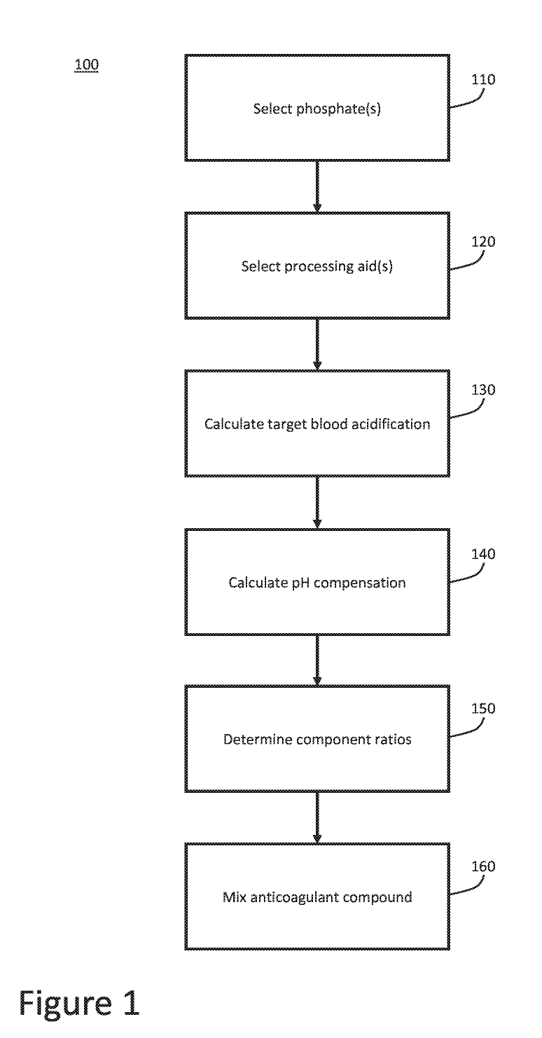

Referring to FIG. 1, example method 100 of making an anticoagulant compound is shown. In 110, at least one phosphate is selected. For example, a polyphosphate such as a pyrophosphate or a tripolyphosphate may be selected for its anticoagulant characteristics and performance in specific pH conditions. Some examples of pyrophosphates may include disodium dihydrogen diphosphate, trisodium diphosphate, tetrasodium diphosphate, tetrapotassium diphosphate, pentasodium triphosphate, pentapotassium triphosphate, sodium polyphosphates, and potassium polyphosphates. In some embodiments, disodium pyrophosphate and sodium tripolyphosphate may be used as phosphates. For example, disodium pyrophosphate may be used in 5-40% dry mix with a selected processing aid. For example, sodium tripolyphosphate may be used in 1-30% dry mix with a selected processing aid. In some embodiments, 10-20% phosphates may be used. In some embodiments, 2-3 different phosphates may be used to balance different functional characteristics in terms of anticoagulant and pH properties. For example, both sodium tripolyphosphate and disodium pyrophosphate may be used in the same anticoagulant compound.

In 120, at least one processing aid is selected. For example, an alkalizing processing aid selected from the alkalizers generally regarded as safe by the U.S. Food and Drug Administration, U.S. Department of Agriculture, or similar regulatory bodies may be selected to assist in increasing the pH of acidic or acidifying animal blood. A processing aid may be defined as an ingredient used for a specific purpose (such as pH adjustment) that has no functional purpose beyond that in the resulting blood product. A variety of processing aids are capable of adjusting pH and used throughout various stages of meat, poultry, and egg processing. For example, an alkalizer may be selected from potassium carbonate, potassium bicarbonate, potassium hydroxide, sodium carbonate, sodium bicarbonate, or sodium hydroxide. In some embodiments, sodium carbonate may be used in 2-90% dry mix with the selected phosphates. Processing aids may be less expensive than phosphates and 80-90% alkalizing processing aid may be effective in some applications.

In 130, the anticipated pH of acidifying animal blood to be treated may be calculated. Anticipated acid level may be based upon the animal, slaughter process, and configuration of blood collection, as well as the application timing and method of the anticoagulant compound and how it intersects with the anticipated degradation of the animal blood. For example, a particular meat processing facility may measure that when processing pigs, the pH of collected blood when it reaches the sprayers for applying anticoagulant are typically at a pH of 5.5.

In 140, the amount of pH compensation to return the treated animal blood to a desired pH range, generally substantially neutral. For example, untreated animal blood may have a pH of 5.5 and the pre-rigor animal may maintain a blood pH around 7.35 in healthy homeostasis. The pH compensation may include adding anticoagulant compound in sufficient concentration and volume to change the pH of the treated blood from the pre-treatment acid level of 5.5 to a pH greater than 7 and less than 7.5. Because the pH compensation generally treats acidic conditions in the blood, the anticoagulant compound will be generally be basic in proportion to the anticipated acid levels. For example, an anticoagulant compound may be formulated to have a pH of 8-8.8 to provide the desired pH compensation. In some embodiments, the water that may be used for applying the anticoagulant compound to the untreated animal blood may also influence the pH compensation. For example, water with slightly basic or slightly acidic characteristics may be selected to reduce or increase the pH compensation desired in the anticoagulant compound. Alternately, the pH of the water for applying the anticoagulant compound may be taken as a given and used to increase or decrease the pH compensation needed for the anticoagulant compound, thus changing the formulation of the anticoagulant compound to compensate for the available water.

In 150, component ratios for the anticoagulant compound may be determined. In a dry mixture, the dry weight of the processing aids and the phosphates may total 100% and be expressed in percentage of dry weight or a weight ratio. For example, a selected anticoagulant compound for a particular application with a desired level of pH compensation may include a ratio of 20% phosphates to 80% alkalizing processing aids. The percentage of phosphates may be provided as a desired percentage with the remaining percentage of alkalizing processing aids making up the balance to 100%. Because the alkalizing characteristics of different alkalizing processing aids may vary, as may the performance characteristics of selected phosphates, the ratios may differ by selected materials and applications. In addition, phosphates may have different stability and interaction with blood constituents at different pH levels, which may further recommend particular ratios and target pH levels for certain applications.

In 160, the anticoagulant compound is mixed. The selected phosphate(s) and alkalizing processing aid(s) are added in their desired ratios and mixed according to a process appropriate for the form and state of the materials. For example, sodium carbonate in powder form may be combined with disodium diphosphate in a desired proportion calculated to raise the pH level of treated animal blood to near pre-rigor levels. The resulting anticoagulant compound may be customized for the application to create superior effects in the stabilization of animal blood (prevention of clotting and degradation of cell walls and other blood constituents) and increasing the yield of desired blood products (produced through further processing of the treated blood). For example, an example anticoagulant compound may include 40% sodium tripolyphosphate, 35% disodium pyrophosphate, and 25% sodium carbonate to achieve a desired anticoagulant compound pH calculated to offset the acidic pH of the acidifying blood in a known application concentration and process. In some embodiments, no other additives may be included in the anticoagulant compound (or any resulting aqueous solution thereof) and the anticoagulant compound may consist entirely of the selected phosphate(s) and selected alkalizing processing aid.

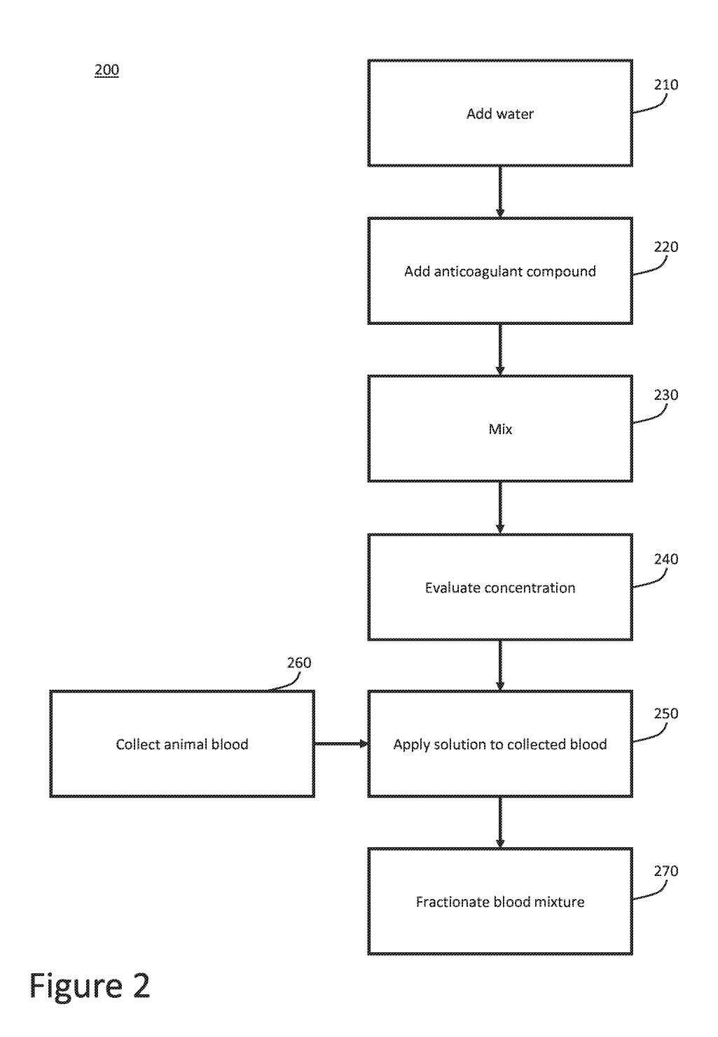

Referring to FIG. 2, an example method 200 of using an anticoagulant compound is shown. For example, the anticoagulant compound made using example process 100 in FIG. 1 may be used in accordance with method 200. Method 200 may be performed within a single meat or poultry processing facility or portions may be completed in different facilities. In some embodiments, method 200 is performed in the context of an automated system for mixing and dispensing stabilization additives, such as anticoagulant compounds, into the blood collection path of a meat processing facility. For example, a meat processing facility may include collection troughs for collecting untreated animal blood from the animals as they are slaughtered that are equipped with pump-activated sprayers for applying anticoagulant compounds into the troughs. Such an automated system may further include hoppers for receiving dry anticoagulant compound and/or tanks for receiving water and/or pre-mixed aqueous solution of anticoagulant compound. A tank or other vessel may be provided for receiving water and anticoagulant compound (dry or high-concentration aqueous solution) and the tank or other vessel may enable transfer to a spray or other dispensing system or may be directly connected by one or more fluid paths and valves.

In 210, water may be added to a tank or other vessel for preparing the anticoagulant compound for application to animal blood. For example, the water may be part of a building water system or come from a separate water storage container or system. The water used may include softened water, well water, municipal water, spring water, distilled water, deionized water, or any other water source for creating and/or diluting an aqueous solution. The water may be added to the tank in order to achieve a desired solution with the anticoagulant compound. For example, water may be added such that the anticipated amount of anticoagulant compound will result in an aqueous solution of 1-60% anticoagulant compound with the balance being water.

In 220, an anticoagulant compound, such as the anticoagulant compound of method 100, may be added to the water in the desired proportion. In some embodiments, the anticoagulant compound may be controllably added to achieve a desired aqueous solution that is 1-60% anticoagulant compound with water making up 40-99% of the solution. The composition of the anticoagulant compound, as well as the solution dispensing rate, solution dispensing volume, and rate and volume of animal blood to be treated may be relevant to calculating the desired concentration of the anticoagulant compound in the solution.

In 230, the anticoagulant compound may be mixed with the water until it goes into solution. For example, an agitator or other mixing apparatus may be present in or inserted into the tank and the anticoagulant compound and water may be agitated for a desired time, such as 10 minutes. In some embodiments, that agitation may be started in the water alone and the water may be agitated during the addition of the anticoagulant compound in 220.

In 240, the resulting solution of anticoagulant compound and water may be evaluated for concentration and/or other characteristics. For example, the solution may be checked for total dissolved solids (TDS) using a refractometer. This may provide an additional quality control to verify that the correct concentration is being used, as well as verifying that the desired dissolved state has been achieved to assure distribution and protect application equipment.

In 250, the solution may be applied to collected animal blood and/or animal blood during the collection process. For example, the solution of anticoagulant compound may be applied to or otherwise mixed with collected animal blood. The solution may be held in a storage tank and then controllably pumped to sprayers at a collection trough. The solution is thereby applied to the collected animal blood. Similar methods may be used for conveying the solution to one or more dispensing points where the anticoagulant compound in solution may be sprayed, drenched, trenched, or otherwise applied to the blood for the purpose of stabilization.

In 260, the animal blood is collected. This may be a prerequisite to applying the solution to the collected blood or the solution may be applied to the carcass or mixed with the blood on its way to collection. Various methods of blood collection, including basins, troughs, or other catch features that may be positioned to receive blood, as well as channels, drains, or conduits that may lead to blood collection tanks or other apparatus, may be part of application process for the solution.

In 270, further processing of the collected and treated blood may be done. For example, the mixture of blood and anticoagulant compound solution may be separated and the blood may be fractionated. In some embodiments, the mixture may be centrifuged to remove water and excess solution and the blood may be fractionated into a cellular fraction and a liquid fraction. The cellular fraction may include predominately red blood cells and may also include the small proportion of white blood cells and platelets. The liquid fraction is predominately plasma and protein components suspended therein. The animal blood processed in this way may yield a greater ratio of liquid fraction to cellular fraction and the liquid fraction may have superior color. For example, the liquid fraction may be at least 53% of the fractionated animal blood, compared to 50% achieved by other methods. In some embodiments, the liquid fraction may be at least 60% of the fractionated animal blood. In some meat or poultry processing, the plasma in the liquid fraction may be a more valuable blood product, particularly if it tends toward translucent white or clear in color.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

What is claimed is:

1. An anticoagulant compound consisting essentially of:
at least one phosphate in a percent dry mix of forty percent or less; and
at least one alkalizing processing aid making up a balance of the anticoagulant compound in proportion to the at least one phosphate;
wherein animal blood to be processed has begun acidification and a concentration of anticoagulant compound is calculated to return post-rigor animal blood to a substantially neutral pH.

2. The anticoagulant compound of claim 1, wherein the at least one phosphate consists essentially of polyphosphates.

3. The anticoagulant compound of claim 2, wherein the at least one phosphate is selected from a pyrophosphate or a tripolyphosphate.

4. The anticoagulant compound of claim 3, wherein the at least one phosphate is selected from disodium pyrophosphate or sodium tripolyphosphate.

5. The anticoagulant compound of claim 1, wherein the at least on alkalizing processing aid is selected from: potassium carbonate, potassium bicarbonate, potassium hydroxide, sodium carbonate, sodium bicarbonate, or sodium hydroxide.

6. The anticoagulant compound of claim 1, wherein the anticoagulant compound is mixed with water to form an aqueous solution between 40-99% water.

7. The anticoagulant compound of claim 6, wherein the aqueous solution is mixed with animal blood as the animal blood is collected for processing.

8. The anticoagulant compound of claim 7, wherein the animal blood is fractionated after collection and yields at least 53% liquid fraction.

9. The anticoagulant compound of claim 7, wherein the animal blood is fractionated after collection and yields at least 60% liquid fraction.

10. A method comprising:
selecting at least one phosphate;
selecting at least one alkalizing processing aid;
mixing the at least one phosphate and the at least one alkalizing processing aid to form an anticoagulant compound, wherein the at least one phosphate is forty percent or less of the anticoagulant compound and the at least one alkalizing processing aid makes up a balance of the anticoagulant compound; and
selecting a concentration of anticoagulant compound calculated to return post-rigor animal blood that has begun acidification to a substantially neutral pH.

11. The method of claim 10, wherein the at least one phosphate consists essentially of polyphosphates.

12. The method of claim 11, wherein the at least one phosphate is selected from a pyrophosphate or a tripolyphosphate.

13. The method of claim 12, wherein the at least one phosphate is selected from disodium pyrophosphate or sodium tripolyphosphate.

14. The method of claim 10, wherein the at least on alkalizing processing aid is selected from: potassium carbonate, potassium bicarbonate, potassium hydroxide, sodium carbonate, sodium bicarbonate, or sodium hydroxide.

15. The method of claim 10, further comprising mixing the anticoagulant compound with water to form an aqueous solution between 40-99% water.

16. The method of claim 15, further comprising mixing the aqueous solution with animal blood as the animal blood is collected for processing and thereby substantially preventing coagulation and neutralizing an acidic pH of the animal blood to a post-rigor pH level of at least 7.

17. The method of claim 16, further comprising fractionating the animal blood into a cellular fraction and a liquid fraction, wherein the liquid fraction is at least 53%.

18. The method of claim 16, further comprising fractionating the animal blood into a cellular fraction and a liquid fraction, wherein the liquid fraction is at least 60%.

19. A method of producing fractionated animal blood in a meat processing facility, comprising:
collecting acidifying animal blood from an animal carcass;
applying an anticoagulant compound to the animal blood, the anticoagulant compound comprising an alkalizing processing aid and a phosphate in an aqueous solution with water, wherein an acidic pH of the animal blood prior to applying the anticoagulant compound is substantially neutralized by the anticoagulant compound; and
fractionating the animal blood into a cellular fraction and a liquid fraction, wherein the liquid fraction is at least 53% of the fractionated animal blood.

20. The method of claim 1, wherein the liquid fraction is at least 60% of the fractionated animal blood.
